(19) United States
(12) Patent Application Publication
(10) Pub. No.: US 2003/0008014 A1
(43) Pub. Date: Jan. 9, 2003

(54) Truncated apolipoprotein B-containing lipoprotein particles for delivery of compounds to tissues or cells

(76) Inventor: Gregory S. Shelness, Winston-Salem, NC (US)

Correspondence Address: MYERS BIGEL SIBLEY & SAJOVEC, PO BOX 37428, RALEIGH, NC, 27627, US

(21) Appl. No.: 09/885,894

(22) Filed: Jun. 20, 2001

(51) Int. Cl. A61K 38/17; A61K 9/50; C07K 14/775

(52) U.S. Cl. 424/499; 514/21; 530/359

(57) ABSTRACT

A lipoprotein compound delivery particle comprises: (a) a lipophilic or amphipathic compound to be delivered (e.g. paclitaxel); (b) at least one polar lipid (this ingredient being optional when the lipophilic or amphipathic compound serves itself as a polar lipid); (c) optionally, at least one neutral lipid; and (d) a truncated apolipoprotein B (apoB) protein having a deleted LDL receptor binding region. Conjugates useful for making such particles, pharmaceutical formulations containing such particles, and methods of using such particles are also described.

### STATEMENT OF FEDERAL SUPPORT

This invention was made possible with government support under grant number HL49373 from the National Institutes of Health. The United States government has certain rights to this invention.

### FIELD OF THE INVENTION

The present invention concerns the use of the lipid sequestering region of ApoB100 as a delivery vehicle of lipophilic active ingredients such as pharmaceutical drugs or diagnostic agents.

### BACKGROUND OF THE INVENTION

Apolipoprotein B (apoB) is a 4536 amino acid protein secretory glycoprotein that is essential for the assembly and secretion of hepatic very low density lipoproteins (VLDL) (Kane and Havel (1995) In: The Metabolic Basis of Inherited Disease. C. R. Scriver, A. L. Beaudet, W. S. Sly, and D. Valle, eds. McGraw-Hill, New York. pg 1139-1163; Homanics et al. (1993) Proc. Natl. Acad. Sci. USA 90:2389-2393). In addition to the ubiquitous protein and lipid biosynthetic and trafficking factors present in most cell types, VLDL assembly require a dedicated cofactor termed, microsomal triglyceride transfer protein (MTP) (Gordon (1997) Curr. Opin. Lipidol. 8:131-137). MTP is expressed predominantly in lipoprotein producing cells (intestine, liver and heart) and may function by transferring lipid from the ER membrane to apoB during its cotranslational translocation into the ER lumen (Gordon (1997) Curr. Opin. Lipidol. 8:131-137). VLDL particles emerge from the liver as 30-80 nm diameter spherical emulsion particles composed of a triglyceride core surrounded by a surface monolayer of phospholipid. VLDL functions to deliver triglyceride to peripheral tissues for metabolism or storage (Havel and Kane (1995) In: The Metabolic Basis of Inherited Disease. C. R. Scriver, A. L. Beaudet, W. S. Sly, and D. Valle, eds. McGraw-Hill, New York. pg 1841-1851). Because of apoB's large size and extreme hydrophobicity, studies of how it, in combination with MTP, recruits and sequesters lipid in the hepatic secretory pathway have been difficult. Nonetheless, this is an area of active investigation as VLDL serves as the metabolic precursor of atherogenic low density lipoproteins (LDL).

Recent studies on apoB assembly have focused on the role of the amino-terminal (α₁) globular domain of apoB in the initiation of lipoprotein formation (FIG. 1). When this domain is expressed in transfected cell on its own (i.e., the amino terminal 17% of apoB; apoB17) it is incapable of forming a lipoprotein particle. However, when the lipid-free form of this protein is incubated with phospholipid vesicles composed of dimyristoylphosphatidylcholine (DMPC), apoB17 solubilizes the lipids forming discoidal recombinant lipoprotein particles (Herscovitz et al. (1991) Proc. Natl. Acad. Sci. USA 88:7313-7317). This strong affinity for lipids may reflect a function that the α₁ domain of apoB plays during the initiation of lipoprotein assembly in vivo (Shelness and Sellers, (2001) Curr. Opin. Lipidol. 12, 151-157; Shelness et al. (1999) J. Nutr. 129 Suppl.:456S-462S). The use of truncated apoB for delivery of compounds to cells, tissues or subjects has neither been suggested nor disclosed.

### SUMMARY OF THE INVENTION

A first aspect of the present is a lipoprotein compound delivery particle. The particle comprises:

(a) a lipophilic or amphipathic compound to be delivered;

(b) at least one polar lipid (this ingredient being optional when the lipophilic or amphipathic compound serves itself as a polar lipid);

(c) optionally, at least one neutral lipid; and

(d) a truncated apolipoprotein B (apoB) protein having a deleted LDL receptor binding region.

The particles may take any of a variety of different physical forms, including but not limited to discoidal particles (particles without a neutral lipid core), small emulsion particles (having a neutral lipid core), large emulsion particles (having a neutral lipid core), etc.

In one embodiment of the foregoing, the apoB further comprises a fused (including covalently conjugated) heterologous moiety, where said heterologous moiety is a member of a specific binding pair such as a receptor binding compound.

A second aspect of the present invention is a pharmaceutical formulation comprising a plurality of lipoprotein compound delivery particles as described above (and further described below).

A third aspect of the present invention is a method of delivering a compound (e.g., a diagnostic agent or theapeutic agent (drug) to a subject in need thereof, comprising administering a lipoprotein compound delivery particle as described above (and below) to said subject in an amount effective to deliver the compound to the subject.

A fourth aspect of the present invention is the use of a particle as described above for the preparation of a medicament for delivering the compound to be delivered to a subject in need thereof.

A fifth aspect of the present invention is a covalent conjugate useful for the preparation of particles as described above. The conjugate comprises a truncated apolipoprotein B protein having a deleted LDL receptor binding region covalently coupled to a heterologous moiety, where the heterologous moiety is a member of a specific binding pair. The conjugate may be provided in any suitable form such as a fusion protein.

A further aspect of the present invention is a truncated apoB protein useful for the production of fragments as described above. Such truncated proteins consist of a continuous segment of mature apoB (preferably mammalian and most preferably human) containing, comprising or consisting of at least amino acids 1 through 861, 884 or 931, but with amino acids 932 and above deleted. Examples include, but are not limited to, apoB19, apoB19.5, and apoB20.5. A heterologous peptide may be fused to such truncated apoB proteins to form a fusion protein as noted above.

A still further aspect of the present invention is nucleic acids (e.g, DNA, RNA) encoding truncated apoB proteins, and fusion proteins, as described above.

In one embodiment, the present invention exploits the avid lipid binding properties of truncated apoB to compartmentalize lipophilic drugs, such as paclitaxel. Paclitaxel is a complex ester that perturbs microtubule depolymerization. As such it arrests cells in the G2 and M phases of the cell cycle. Paclitaxel is commonly used to treat breast and other carcinomas; however, its lipophilic properties necessitates intravenous (iv) administration as a caster oil/ethylene oxide emulsion (cremophor) containing 50% ethanol. Indeed, the vehicle may cause many of the toxic side effects of paclitaxel (Beck et al. (1997) In: Cancer Medicine, Volume One. J. F. Holland, R. C. Bast, Jr., D. L. Morton, E. Frei, III, D. W. Kufe, and R. R. Weichselbaum, eds. Williams & Wilkins, Baltimore. pg. 1005-1025). As such, the compartmentalization of paclitaxel into small polar lipid complexes using a subdomain of an abundant, endogenous human plasma protein may represent an inherently more benign formulation.

Chemotherapeutic drugs are often toxic at concentrations necessary to eradicate or attenuate a particular disease state. In part this is because drugs are delivered systemically, even though only one or a small subset of cell or tissue types is actually affected. Furthermore, some drugs are lipophilic and must be delivered to patients as emulsions containing oils and/or polar organic solvents, often producing toxic side effects. This invention describes the creation of a protein-based drug delivery system based on sequences contained within apolipoprotein B100 (apoB100). This protein can sequester lipophilic drugs in recombinant lipoproteins and can be modified in a number of ways to control the type of particle generated, the lipids and drugs it associates with, and the cells to which it can be targeted.

Monoclonal antibodies are currently being used as therapeutic agents. If the antibody interacts with a specific cell surface protein that is critical for the growth and proliferation of the cell, such as the HER2/neu oncogene over-expressed in some mammary tumor cells, its function can be attenuated, resulting in reduced cellular proliferation. However, depending upon the cell surface marker, antibody binding on its own is not necessarily therapeutic. This invention combines cell specific targeting that can be achieved with antibodies, ligands, or receptors with the delivery of chemotherapeutic lipophilic agents (including water-soluble compounds that are modified to confer properties of either polar or neutral lipids).

To an extent, targeted liposomes are an attempt to achieve a similar end. In this approach, liposomal vesicles are prepared containing a therapeutic drug either in the aqueous interior of the liposome or dissolved within the lipid bilayer. The targeting of the liposome requires covalent attachment of a targeting protein such as an antibody or peptide ligand. However, these methods require chemical conjugation of the protein to the liposomal lipids, a process that is not always efficient and does not always orient the protein in a predictable fashion. Furthermore, liposomes possess limited capacity for dissolution of lipophilic drugs in their bilayer surfaces.

Another problem with liposomes is their rapid clearance by the reticular endothelial system (RES). Hence, liposomes often display very short plasma half-lives. To circumvent this problem, the surface of liposomes are covalently or noncovalently modified. These treatments, however, often limit the ability of the liposome to interact with the target tissue. While it is not clear whether the recombinant lipoprotein particles described in this invention have longer half-lives than liposomes, the use of apoB and all of its possible truncation mutants and variants, combined with the capacity to use various molecular species of lipid including synthetically modified lipids already used to create RES evading (stealth) liposomes, will provide the opportunity to produce bifunctional lipoproteins with very different properties, some of which may include relatively prolonged plasma half-lives.

It should be noted that low density lipoprotein (LDL), the most abundant form of apoB100-containing lipoproteins present in human plasma, has been used to selectively concentrate lipophilic photosensitizers. LDL are emulsion particles containing a neutral lipid core composed primarily of cholesteryl ester and a single molecule of apoB100. The selective uptake of LDL is based on up-regulation of LDL receptors in some transformed cells. Although LDL has already been used to target drugs to cells expressing LDL receptor, the present invention proposes to use truncated forms of apoB lacking the domain(s) that interact with LDL receptor. Hence, these recombinant lipoproteins will contain no intentional targeting information and can, therefore, be directed to other cells and tissues based on genetic fusion with appropriate targeting peptides.

In one embodiment the method described herein expresses truncated apoB as a fusion protein with a single chain antibody, ligand or receptor, or any other peptide sequence that would be specific for a particular macromolecular cell surface marker (Targeting Peptide; FIG. 2, A). Depending on the length of apoB expressed, the type of lipoprotein particle generated can differ. It has already been shown that apoB17 is capable of sequestering lipid in the form of discoidal lipoprotein particles (FIG. 2, C). These particles are basically small phospholipid bilayers whose edges are stabilized by the apolipoprotein. These particles can be generated by direct incubation of apoB17 with certain types of phospholipid vesicles or by a reconstitution process involving cholate dialysis. Incubation of lipophilic compounds with polar lipids during the reconstitution process will likely result in their incorporation into recombinant discoidal lipoproteins. For example, cholesterol is readily packaged within discs formed by chlorate dialysis using apolipoprotein AI. Hence, similarly lipophilic compounds of therapeutic or medical relevance can also be sequestered within these types of particles.

If longer forms of apoB are expressed (e.g., apoB21 and higher), they tend to form spherical lipoproteins containing a neutral lipid core (FIG. 2, B). The size of these particles is roughly proportional to the length of apoB expressed. Hence, particle diameter can be readily controlled. The assembly and secretion of these emulsion particles in cells requires a dedicated cofactor termed microsomal triglyceride transfer protein (MTP). MTP is generally expressed in cells that also express apoB, namely, liver intestine and heart. However, if apoB is coexpressed with MTP, these emulsion particles are generated in virtually all cells tested. Therapeutic or other medically relevant compounds could be sequestered in the core of these particles by a variety of methods. First, if these emulsion-type lipoproteins are incubated with lipophilic compounds added in the appropriate solvent, one may see the spontaneous partitioning of the compound into the existing lipophilic core. Alternatively, reconstitution of apoB100-containing lipoproteins with polar and/or neutral lipids has been achieved. Polar and apolar compounds could be similarly reconstituted into recombinant lipoprotein emulsions formed with any form of apoB that has been truncated and/or has been fused to a targeting sequence. In one related example, human LDL was reconstituted such that the neutral lipid core was replaced with the lipophilic photosensitizer, 1-pyrenemethyl-23,24-bisnor-5-cholen-22-oate-3β-yl oleate (PCO) (Mosely et al. (1981) Proc. Natl. Acad. Sci. USA 78:5717-5721).

Once the recombinant drug-containing lipoproteins are produced, they can be used to deliver their contents to specific cell types depending on the nature of the targeting sequence that is added to the apoB sequence under study (FIG. 2, A). One specific example of the application described herein is a fusion between apoB15 or apoB17 and a single chain antibody directed against the HER-2/neu (HER-2) oncogene. HER-2 is a cell surface protein that is overexposed in certain breast and other cancers. The interaction between the anti-HER-2 antibody and the HER-2 oncogene product would result in selective delivery of the recombinant protein and its associated lipids and drugs to HER-2-overexpressing cells. This delivery of drug to the HER-2 expressing cells could occur by endocytosis, phagocytosis, pinocytosis, bulk fluid phase uptake, or by a selective transfer of drug into the cells by virtue of the close apposition of the recombinant particle and the cell surface. This arrangement may provide high efficiency targeting of drugs to target cells. As a result, the effective concentration of the drug may be enhanced at the disease site while the overall systemic toxicity of the compound reduced.

It should also be noted that simultaneous administration of paclitaxel, a lipophilic anti-cancer drug) and anti HER-2 antibody (Herceptin™) displays greater efficacy than either agent administered alone. The design of a single macromolecular species that achieves both Herceptin™ function and the cell-specific delivery of paclitaxel contained within a conjugated recombinant lipoprotein may display similar or enhanced therapeutic efficacy at considerably lower concentrations of paclitaxel than currently prescribed. While this approach is described herein for cell-type specific delivery of drug to HER-2 overexpressing tumor cells, the approach is a general one that may have broad applicability to numerous forms of cancer and other diseases that are accompanied by cell surface expression of specific protein markers.

The foregoing and other objects and aspects of the present invention are explained in detail in the specification set forth below.

### BRIEF DESCRIPTION OF THE DRAWINGS

FIG. 1 depicts the domain structure of apoB. ApoB has been described in terms of a pentapartite structure in which three amphipathic α-helical domains (α₁, α₂, and α₃) alternate with two domains predicted to form amphipathic β-sheet structure (β₁ and β₂) (Segrest, et al. (1994) Arterioscler. Thromb. 14:1674-1685). The α₁ domain is unique as it possesses both amphipathic lipid binding properties and a globular character, in part due to its high density of intramolecular disulfide bonds (Yang et al. (1990) Proc. Natl. Acad. Sci. USA 87:5523-5527) (bracketed numbers indicate the position of cysteine residues involved in disulfide bond formation; numbers to left of brackets indicate N to C terminal numbering of disulfide bonds). Experiments have demonstrated that folding of the globular α₁ domain is critical to initiate apoB-containing lipoprotein assembly (Shelness and Thornburg (1996) J. Lipid Res. 37:408-419; Ingram and Shelness (1997) J. Biol. Chem. 272:10279-10286).

FIG. 2 depicts the basic concept of targeted drug complex delivery using an apoB fusion protein specific for a particular macromolecular cell receptor. The “X” in apoB“X” refers to the different lengths of apoB that are expressed depending on the type of lipoprotein particle characteristics desired.

FIG. 3 depicts ApoB and single-chain antibody fusion constructs. ApoB17F contains the amino-terminal 17% of apoB (FIG. 1) followed by the 8-amino acid FLAG tag (DYKDDDDK; SEQ ID NO: 1). ApoB17F/αHER2 contains apoB17F followed by anti-HER2 single-chain antibody (Batra, et al. (1992) Proc. Natl. Acad. Sci. USA 89:5867-5871). αHER2F is a control construct in which the anti-HER2 single-chain antibody was engineered with the bovine preprolactin signal peptide (SP) at its N-terminus and the FLAG peptide at its C-terminus.

FIG. 4 depicts various lipid and lipid-paclitaxel discoidal complexes formed with apoB17F and apoB17F/αHER2. Phospholipid and paclitaxel are solubilized with sodium cholate. After addition of apoB17F (Panel A) or apoB17F/αHER2 (Panel B), cholate is removed by dialysis. In the absence of disk forming protein, the phospholipid would form vesicles (liposomes). In the presence of apoB17F, recombinant discoidal complexes are formed. Each particle will likely contain two molecules of apoB17 or apoB17/αHER2 (Herscovitz, et al. (1991) Proc. Natl. Acad. Sci. USA 88:7313-7317).

FIG. 5 shows the expression and purification of apoB17F. Supernatant (250 mL) from baculovirus infected Sf9 cells was subjected to M2-agarose immunoaffinity chromatography (2 mL bed volume). After the last column wash, apoB17F was eluted by sequential rounds of batch incubation with FLAG peptide (lanes 5-7). One mL (0.4%) of the Load (L), flow-through (FT) and final wash fraction and 20 μL (1%) of each eluted fraction was subjected to SDS-PAGE and Coomassie staining. Lanes 8 and 9 contain 2 and 3 μg, respectively of bovine serum albumin. The arrow indicates the position of apoB17F.

FIG. 6 shows the phospholipid disk forming capacity of apoB17F. In panel A, apoB17F (200 μg/ml) was incubated with (+) and without (−) 4 mg/mL DMPC multilamellar vesicles for 20 h. Density gradient centrifugation was used to separate the lipid poor bottom (B) fraction from the lipid-containing Top (T) fraction. The fractions were analyzed by SDS-PAGE and Coomassie staining. As observed, apoB17F binds to DMPC vesicles and is, therefore, recovered exclusively in the top fraction (compare lanes 1 and 2 to lanes 3 and 4). In panel B, the optical density of the vesicle binding assay was monitored. Without addition of apoB, the DMPC vesicle suspension displays a high optical density due to light scattering. Upon addition of apoB17F, the vesicles are “solubilized” by apoB and form small discoidal lipoproteins. Hence, the optical density is reduced to background levels within the 20 h incubation period.

FIG. 7 shows the expression of apoB17F/αHer2 fusion protein in transfected COS cells. COS cells were transfected with apoB17F/αHER2 fusion protein construct (FIG. 3). Twenty-four hours post-transfection, cells were labeled for 2 h with [³⁵S] Met. Cell lysate (C) and media (M) were immunoprecipitated with anti FLAG (M2) antibody and protein G-Sepharose. Immune complexes were analyzed by SDS-PAGE and fluorography. The arrow indicates the position of the fusion protein

FIG. 8 shows electrospray mass spectrometry of paclitaxel. A 100 nM solution of paclitaxel was incubated with LiOH and 100 μL was injected. The spectrum was scanned for parents of m/z 292, a characteristic fragmentation product of the 860.56 paclitaxel parent ion.

FIG. 9 depicts the structure of the apoB23 anti-HER2 single-chain antibody fusion protein (HB23αH2). Note that the diagram is not drawn to scale.

FIG. 10 shows and SDS-PAGE gel of radiolabeled proteins isolated from cells overexpressing HB23αH2 fusion protein. Lanes 1 and 2, proteins from cell (C) and media (M) were immunoprecipitated with anti-apoB antibodies. Lanes 3-7 is a media sample subjected to nickel chromatography. Lane 3, aliquot of media prior to nickel chromatography (L); Lane 4, material that failed to bind to nickel column (FT); Lane 5, last wash of affinity column (W); Lane 6, first elution with imidazole (E1); Lane 7, second elution with imidazole, (E2). Proteins were visualized by fluorography.

FIG. 11 shows proteins isolated from a recombinant baculovirus harboring the apoB23 anti-HER2 single-chain antibody fusion protein gene diagrammed in FIG. 9. Panel A shows the results of Coomassie staining. Panel B is an immunoblot probed with sheep, anti-human apoB antibody. Early viral supernatant was used to infect fresh Sf9 cells with the volumes indicated. Viral supernatants (1 mL for Panel A and 0.25 mL for Panel B) were precipitated with trichloroacetic acid and fractionated by SDS-PAGE. Proteins were visualized by chemiluminescence. MW, molecular weight standards; apoB17H, control baculovirus supernatant containing C-terminally His-tagged form of apoB17.

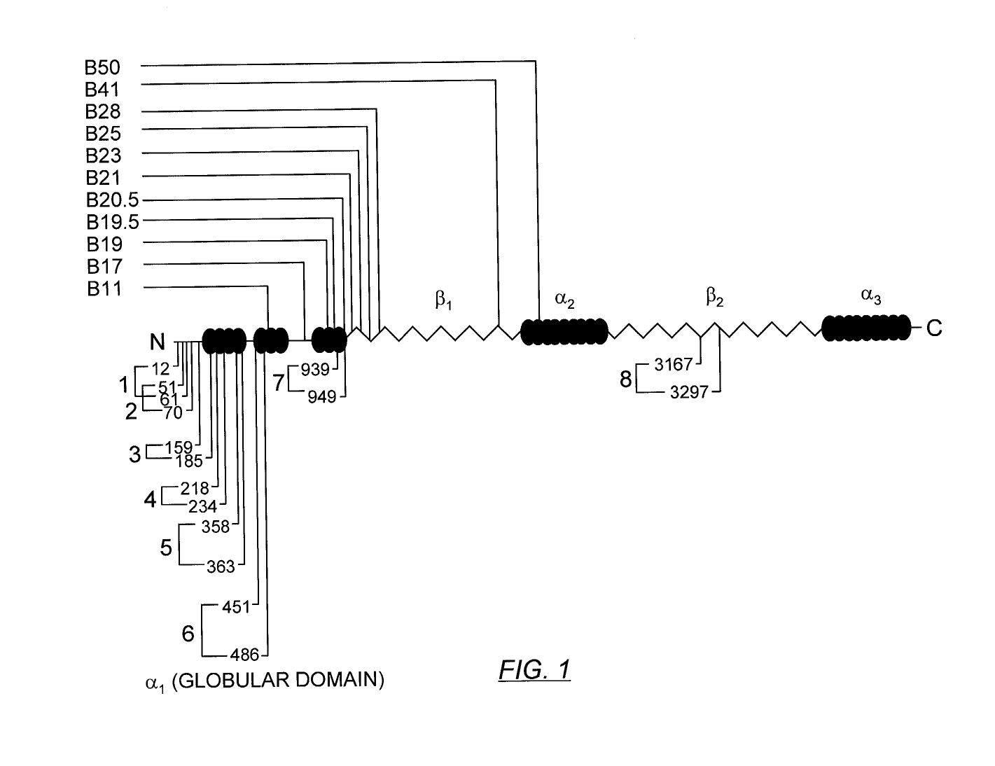

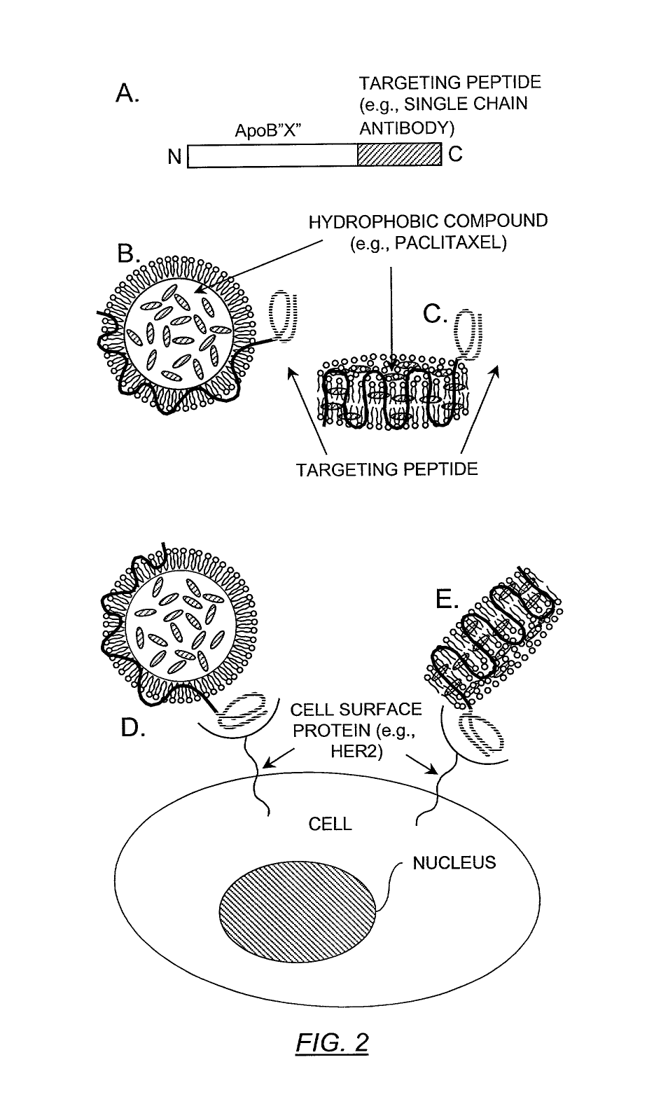

### DETAILED DESCRIPTION OF PREFERRED EMBODIMENTS

The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise.

Except as otherwise indicated, standard methods may be used for the production of cloned genes, expression cassettes, vectors, proteins and protein fragments, and transformed cells according to the present invention. Such techniques are known to those skilled in the art (see e.g., SAMBROOK et al., EDS., MOLECULAR CLONING: A LABORATORY MANUAL 2d ed. (Cold Spring Harbor, N.Y. 1989); F. M. AUSUBEL et al, EDS., CURRENT PROTOCOLS IN MOLECULAR BIOLOGY (Green Publishing Associates, Inc. and John Wiley & Sons, Inc., New York).

Applicants specifically intend that all United States patent references cited herein be incorporated herein by reference in their entirety.

1. Compounds to be Delivered.

The term “lipophilic compound” or “lipophilic drug” is defined as a compound or drug which in its non-ionized form is more soluble in lipid or fat than in water. Examples of lipophilic compounds include, but are not limited to, acetanilides, anilides, aminoquinolines, benzhydryl compounds, benzodiazepines, benzofurans, cannabinoids, cyclic peptides, dibenzazepines, digitalis gylcosides, ergot alkaloids, flavonoids, imidazoles, quinolines, macrolides, naphthalenes, opiates (or morphinans), oxazines, oxazoles, henylalkylamines, piperidines, polycyclic aromatic hydrocarbons, pyrrolidines, pyrrolidinones, stilbenes, sulfonylureas, sulfones, triazoles, tropanes, and vinca alkaloids.

Pharmaceutically active lipophilic drugs which may be incorporated into targeted drug delivery complexes of the invention include drugs for the treatment of cancer and glaucoma, immunoactive agents, antineoplastic agents, anticholinergic and cholinomimetic agents, antimuscarinic and muscarinic agents, antiadrenergic and antiarrhythmics, antihypertensive agents, anti-inflammatory drugs, antibiotic drugs, anti-fungal drugs, steroids, anti-histamines, anti-asthmatics, sedatives, anti-epileptics, anesthetics, hypnotics, antipsychotic agents, neuroleptic agents, antidepressants, anxiolytics, anti-convulsant agents, neuron blocking agents, narcotic antagonists, analgesics, anti-proliferative agents, anti-viral drugs, hormones, and nutrients.

Examples of anti-cancer drugs include but are not limited to paclitaxel, docosahexaenoic acid (DHA)-paclitaxel conjugates, betulinic acid, and doxorubicin (see, e.g. U.S. Pat. No. 6,197,809 to Strelchenok).

Examples of anti-glaucoma drugs include but are not limited to β-blockers such as timolol-base, betaxolol, atenolol, livobunolol, epinephrine, dipivalyl, oxonolol, acetazolamide-base and methzolamide.

Examples of anti-inflammatory drugs include but are not limited to steroidal drugs such as cortisone and dexamethasone and non-steroidal anti-inflammatory drugs (NSAID) such as piroxicam, indomethacin, naproxen, phenylbutazone, ibuprofen and diclofenac acid. Examples of anti-asthmatics include but are not limited to prednisolone and prednisone. (See also U.S. Pat. No. 6,057,347).

An example of an antibiotic drug includes but is not limited to chloramphenicol. Examples of anti-fungal drugs include but are not limited to nystatin, amphotericin B, and miconazole. Examples of an anti-viral drug includes but is not limited to Acyclovir™ (Glaxo Wellcome, U.K.).

Examples of steroids include but are not limited to testosterone, estrogen, and progesterone. Examples of anti-allergic drugs include but are not limited to pheniramide derivatives. Examples of sedatives include but is not limited to diazepam and propofol.

Compounds to be delivered that are not ordinarily lipophilic may be used in the present invention by fusing or covalently coupling them to a lipophilic molecule to produce an amphiphathic compound, as noted further below.

Nucleic acids may be delivered as “lipophilic” compounds by complexing the nucleic acid with a cationic lipid to form a lipophilic complex which can then be incorporated into the neutral lipid core of the particle.

2. Lipids.

The “at least one polar lipid” that may be used to form the complex may include relatively rigid varieties, such as sphingomyelin, or fluid types, such as phospholipids having unsaturated acyl chains (see, e.g., U.S. Pat. No. 6,224,794 to Amsden et al.; U.S. Pat. No. 6,217,886 to Onyuksel et al.; and U.S. Pat. No. 6,200,598 to Needham). Additional examples include but are not limited to phosphatidylcholine, phosphatidylethanolamine, phosphatidylserine, phosphatidylinositol, sphingomyelin, glycosphingolipid, lysolipids thereof, and combinations thereof.

Any suitable neutral lipid may be used to carry out the present invention, including but not limited to triglycerides, cholesterols, derivatives thereof (such as cholesterol esters) and mixtures thereof.

The neutral lipid(s) and/or polar lipid(s) may be replaced, in whole or in part, by the active compound when the active compound is itself a natural or synthetic lipid. Lipids may be the compound to be delivered when the compound to be delivered is for topical application, as in a skin care compound. Numerous additional examples of such active compounds are known, including but not limited to those described in Piantadosi et al., Ether Lipid-Nucleoside Covalent Conjugates, U.S. Pat. No. 5,512,671; and Piantadosi et al., Quaternary Amine Containing Ether or Ester Lipid Derivatives and Therapeutic Compositions, U.S. Pat. No. 5,614,548.

3. Apolipoprotein B.

Apolipoprotein B used to carry out the present invention is typically mature apoB. The apoB may be produced by any suitable means, such as naturally occuring apoB or transgenic or recombinant apoB. The apoB may be of any species but is typically mammalian apoB (e.g., dog, cat, horse, goat) and in one preferred embodiment is human apoB.

The apoB used in the present invention is a truncated apoB that has a deleted LDL receptor binding domain (that is, the receptor binding domain may be wholly or partly deleted, so long as it is inoperative as a receptor binding domain for its ordinary purpose). As is well established in the art, truncated apoB proteins are represented by a centile system, where the number represents the percent of the N terminal fragment remaining (in number of amino acids) after the C terminal deletion, as compared to the full length mature apolipoprotein B. Thus, apoB100 refers to full length mature apoB; apoB50 refers to the N terminal 50% of mature apoB with the C terminal 50% deleted; apoB25 refers to the N terminal 50% of mature apoB with the C terminal 75% deleted; etc. In general, apoB used to carry out the present invention consists of those apoB fragments ranging in size from apoB6 (in general, N-terminal fragments having amino acid numbers 272 and above deleted) apoB8, apoB10, apoB12, apoB 15, apoB19 or apoB19.5 (amino acid numbers 884 and above deleted) up to apoB69.4 (fragments having amino acids 3147 and above deleted), or even through apoB74 (fragments having amino acid 3359 and above deleted).

In the apoB fragments described herein, up to 5, 10, or 20 amino acids or more can optionally be deleted from the N terminus, so long as the ability to form particles is retained.

4. Heterologous Moieties.

Some embodiments of the invention utilize a heterologous moiety. “Targeting (or heterologous) moiety” refers to any material or substance which may promote targeting of tissues and/or receptors in vitro or in vivo with the compositions of the present invention. The targeting moiety may be synthetic, semi-synthetic, or naturally-occurring. The targeting moiety may be a protein, peptide, oligonucleotide, or other organic molecule. The targeting moiety may be an antibody (this term including antibody fragments and single chain antibodies that retain a binding region or hypervariable region). Materials or substances which may serve as targeting moieties include, but are not limited to:

| Targeting Moiety | Target(s) |
|---|---|
| Antibodies (and fragments such as Fab, | RES system |
| F(ab)′2, Fv, Fc, etc.) | |
| Epidermal growth factor (EGF) | Cellular receptors |
| Collagen | Cellular receptors |
| Gelatin | Cellular receptors |
| Fibrin-binding protein | Fibrin |
| Plasminogen activator | Thrombus |
| Urokinase inhibitor | Invasive cells |
| Somatostatin analogs | Cellular receptors |
| Lectin (WGA) | Axons |
| f-Met-Leu-Phe | Neutrophils |
| Selectin active fragments | Glycosyl structures |
| ELAM, GMP 140 | Leucocyte receptors |
| “RGD” proteins | Integrins, Granulocytes |
| IL-2 | Activated T-cell |
| CD4 | HIV infected cells |
| Cationized albumin | Fibroblasts |
| Carnitine | |
| Acetyl-, maleyl-proteins | Macrophage scavenger receptor |
| Hyaluronic acid | Cellular receptors |
| Lactosylceramide | Hepatocytes |
| Asialofoetuin | Hepatocytes |
| Arabinogalactan | Hepatocytes |
| Galactosylated particles | Kupffer cells |
| Terminal fucose | Kupffer cells |
| Mannose | Kupffer cells, macrophages |
| Lactose | Hepatocytes |
| Dimuramyl-tripeptide | Kupffer cells, macrophages |
| Fucoidin-dextran sulfate | Kupffer cells, macrophages |
| Sulfatides | Brain |
| Glycosyl-steroids | |
| Glycosphyngolipids | Other glycosylated structures |
| Hypoxia mediators | Infarcted tissues |
| Amphetamines | Nervous system |
| Barbiturates | Nervous system |
| Sulfonamides | |
| Monoamine oxidase inhibitor | Brain |
| substrates | |
| Chemotactic peptides | Inflammation sites |
| Muscarine and dopamine receptor | Nervous system |
| substrates | |

Also to be included are organ or tissue “homing molecules” as disclosed in U.S. Pat. No. 6,232,287 to Ruoslahti, et al.

5. Conjugates.

Some embodiments of the invention as noted above employ covalent conjugates of the apoB and a targeting moiety. The moiety is not required where targeting of the particle is not desired, and the apoB is utilized in the particle for another purpose such as stabilizing the particle, controlling the size of the particle, facilitating the production of small particles (e.g., particles 18, 15 or 10 nanometers in diameter or less), etc.

When employed, the conjugates may be produced by any suitable means, such as the expression of a fusion protein or by coupling with a synthetic organic chemical reaction. In general, covalent conjugates of the invention comprises(a) a truncated apolipoprotein B protein in having a deleted LDL receptor binding region, as described above, covalently coupled to (b) a heterologous moiety, where said heterologous moiety is a member of a specific binding pair, as described above. In a particular embodiment the conjugate may be a fusion protein, but the conjugates may also be produced by synthetic organic chemical reaction as noted above.

Nucleic acids encoding truncated apoB as described above and fusion proteins as described herein, along with cells containing and expressing such nucleic acids) may be produced in accordance with known techniques.

6. Particles and Formulations.

As noted above, a lipoprotein compound delivery particle of the present invention typically comprises

(a) a lipophilic or amphipathic compound to be delivered (typically in an amount of from 0.05, 0.1 or 1 to 90 or 95 percent by weight);

(b) at least one polar lipid in an amount sufficient to form a particle with said lipophilic compound (typically in an amount of from 0, 0.1 or 1 to 50, 60 or 70 percent by weight)

(c) from 0 to 90 percent by weight of at least one neutral lipid (typically in an amount of from 0, 0.1, or 1 to 90 or 95 percent by weight); and

(d) from 0.5 to 90 percent by weight of a truncated apolipoprotein B protein in said particle having a deleted LDL receptor binding region (typically in an amount of from 0.1, 0.5 or 1 to 90 or 95 percent by weight). The apolipoprotein B may as noted above further comprises a fused or conjugated heterologous moiety, where said heterologous moiety is a member of a specific binding pair. Also, the polar and/or neutral lipids may optionally be replaced, in whole or in part, by the compound to be delivered when the compound to be delivered is a lipid or amphipathic molecule.

In certain embodiments, the particle has a diameter less than about 15, 18 or 20 nanometers. For such particles the truncated apoB facilitates particle formation. In general, however, the particles may have a diameter of from about 3 or 5 to 5,000 nanometers, or more.

While a variety of different apoB truncations may be used to form the particle, as noted above, the apoB fragments comprising at least apoB19.5 are preferred when the particle has a neutral core.

An advantage of the present invention is that the incorporation of apoB into the particles facilitates the formation of particle populations (e.g., incorporated into a pharmaceutical formulation) of substantially uniform size. Recognizing that any population or group of particles will generally be a bell-shaped or gaussian distribution, the present invention, particles of the present invention may be provided in a population or formulation: consisting essentially of said particles in a size of 2 or 5 to 10 or 20 nanometers in diameter; consisting essentially of particles in a size of 5 or 10 to 20 or 40 nanometers in diameter; consisting essentially of particles in a size of 10 or 20 to 30 or 60 nanometers in diameter; consisting essentially of particles in a size of 15 or 30 to 50 or 100 nanometers in diameter; consisting essentially of particles in a size of 25 or 50 to 100 or 200 nanometers in diameter; consisting essentially of particles in a size of 50 or 100 to 500 or 1,000 nanometers in diameter; consisting essentially of particles in a size of 250 or 500 to 5,000 nanometers in diameter, or more.

The composition of the particles may vary depending upon (among other things) the size of the particles. Thus, in some embodiments of the invention, including but not limited to discoidal particles, the composition of the particle may be:

(a) from 0.05, 0.1 or 0.5 to 4, 50 or 60 percent by weight of said compound to be delivered;

(b) from 5, 10 or 20 to 40, 50 or 60 percent by weight of said at least one polar lipid;

(c) from 0, 1 or 2 to 5, 10 or 20 percent by weight of at said least one neutral lipid; and

(d) from 40 or 50 to 70, 80, or 90 percent by weight of said truncated apoB.

In other embodiments of the invention, including but not limited to small emulsion particles (e.g., particles having a diameter less than 25, 20 or 10 nanometers), the composition of the particle may be:

(a) from 0.05, 0.1, or 0.5 to 45, 55 or 65 percent by weight of said compound to be delivered;

(b) from 5, 15 or 25 to 45, 55 or 65 percent by weight of said at least one polar lipid;

(c) from 1, 2 or 3 to 20, 30 or 40 percent by weight of at said least one neutral lipid; and

(d) from 20, 30 or 40 to 70, 80 or 90 percent by weight of said truncated apoB.

In still other embodiments of the invention, including but not limited to large emulsion particles (e.g., particles having a diameter greater than 25, 50 or 100 nanometers) the particle may comprise:

(a) from 0.05, 0.1 or 0.5 to 70, 80 or 90 percent by weight of said compound to be delivered;

(b) from 0.5, 1 or 2 to 20, 30 or 40 percent by weight of said at least one polar lipid;

(c) from 20, 30 or 40 to 80, 90 or 95 percent by weight of at said least one neutral lipid; and

(d) from 0.01, 0.5 or 1 to 5, 10 or 20 percent by weight of said truncated apoB.

As noted above, the particles of the present invention may be provided in a physiologically or pharmaceutically acceptable carrier, or may be provided in a lyophilized form for subsequent use. The compositions are optionally sterile when intended for parenteral administration or the like, but need not always be sterile when intended for some topical application. Any pharmaceutically acceptable carrier may be used, including but not limited to aqueous carriers. Aqueous carriers for parenteral injections include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

7. Methods of Use.

The invention further provides methods of delivering a lipophilic compound, diagnostic agent or drug to a subject, target tissue, or organ comprising the steps of preparing a pharmaceutical formulation comprising a lipophilic drug in association with a lipid in an amount sufficient to form a complex with said lipophilic drug according to the methods of the invention and administering a therapeutically effective amount of the pharmaceutical formulation to said target tissue. The pharmaceutical formulation of the invention may be administered intravenously, intraarterially, intranasally such as by aerosol administration, nebulization, inhalation, or insufflation, intratracheally, intra-articularly, orally, transdermally, subcutaneously, or topically.

By “effective” or “therapeutically effective” amount is meant an amount that relieves (to some extent) one or more symptoms of the disease or condition in the patient. Additionally, by “therapeutically effective amount” is meant an amount that returns to normal, either partially or completely, physiological or biochemical parameters associated with or causative of a condition. Additionally, the effective amount may be one sufficient to achieve some other intended purpose, such as delivery of a radioimaging agent or other diagnostic agent to an organ or tissue.

Still further, the present invention provides a method of identifying a selected organ or tissue or diagnosing a pathology in a selected organ or tissue comprising the steps of preparing a pharmaceutical formulation comprising an appropriate targeting moiety and a diagnostic agent in association with a lipid in an amount sufficient to form a particle with said diagnostic agent according to the methods of the invention and administering to a subject a pharmaceutical formulation to said target organ or tissue.

“Diagnostic agent” refers to any agent which may be used in connection with methods for imaging an internal region of a patient and/or diagnosing the presence or absence of a disease in a patient. Exemplary diagnostic agents include, for example, radioactive and fluorescent labels and contrast agents for use in connection with ultrasound imaging, magnetic resonance imaging or computed tomography imaging of a patient. Diagnostic agents may also include any other agents useful in facilitating diagnosis of a disease or other condition in a patient, whether or not imaging methodology is employed.

The present invention also provides a method of treating a subject suffering from a disorder selected from the group consisting of skin cancer, psoriasis, acne, eczema, rosacea, actinic keratosis, seborrheic dermatitis, and congenital keratinization disorders, in which any composition according to the methods of the invention is administered to the subject in need of such treatment by means of topical application.

The particles of the present invention can, as noted above, be used for topical application. Thus the present invention further provides a method of treating one or more conditions of the skin selected from the group consisting of dry skin, photodamaged skin, age spots, aged skin, increasing stratum corneum flexibility, wrinkles, fine lines, actinic blemishes, skin dyschromias, and ichthyosis, comprising applying to the skin having said one or more condition any composition according to the methods of the invention, where the compound to be delivered is a known compound for treating such conditions, and is delivered in its known amount for treating such conditions. The term “topical application”, as used herein, means to apply or spread the compositions of the present invention onto the surface of the skin.

The compound to be dellivered in topical compositions of the present invention may comprise skin active ingredients. Non-limiting examples of such skin active ingredients include vitamin B3 compounds such as those described in PCT application WO 97/39733, published Oct. 30, 1997, to Oblong et al., herein incorporated by reference in its entirety; flavonoid compounds; hydroxy acids such as salicylic acid; exfoliation or desquamatory agents such as zwitterionic surfactants; sunscreens such as 2-ethylhexyl-p-methoxycinnamate, 4,4′-t-butyl methoxydibenzoyl-methane, octocrylene, phenyl benzimidazole sulfonic acid; sun-blocks such as zinc oxide and titanium dioxide; anti-inflammatory agents; anti-oxidants/radical scavengers such as tocopherol and esters thereof; metal chelators, especially iron chelators; retinoids such as retinol, retinyl palmitate, retinyl acetate, retinyl propionate, and retinal; N-acetyl-L-cysteine and derivatives thereof; hydroxy acids such as glycolic acid; keto acids such as pyruvic acid; benzofuran derivatives; depilatory agents (e.g., sulfhydryl compounds); skin lightening agents (e.g., arbutin, kojic acid, hydroquinone, ascorbic acid and derivatives such as ascorbyl phosphate salts, placental extract, and the like); anti-cellulite agents (e.g., caffeine, theophylline); moisturizing agents; anti-microbial agents; anti-androgens; and skin protectants. Mixtures of any of the above mentioned skin actives may also be used. A more detailed description of these active ingredients is found in U.S. Pat. No. 5,605,894 to Blank et al. Preferred skin active ingredients include hydroxy acids such as salicylic acid, sunscreen, antioxidants and mixtures thereof. Topical applications can be practiced by applying a composition of the invention in the form of a skin lotion, cream, gel, emulsion, spray, conditioner, cosmetic, lipstick, foundation, nail polish, or the like which is intended to be left on the skin for some esthetic, prophylactic, therapeutic or other benefit.

The examples, which follow, are set forth to illustrate the present invention, and are not to be construed as limiting thereof. The embodiment shown in the examples demonstrate that the lipid and drug sequestration activity of the amino terminal 17% of apoB (apoB17) can be used to compartmentalize polar lipids and paclitaxel, a drug commonly used in the treatment of breast carcinomas (Beck et al (1997) ) In: Cancer Medicine, Volume One. J. F. Holland, R. C. Bast, Jr., D. L. Morton, E. Frei, III, D. W. Kufe, and R. R. Weichselbaum, eds. Williams & Wilkins, Baltimore. pg. 1005-1025). The protein was modified by fusion of a single-chain antibody directed against the tumor marker HER2/neu (HER2). HER2-positive breast tumors are commonly treated with combination of an anti-HER2 monoclonal antibody (Herceptin™) and paclitaxel (Slamon et al. (1998) Proc. Am. Soc. Clin. Oncol. 17:98a (abstract)). The design of a single macromolecular species that achieves both Herceptin™ function and cell-specific delivery of a paclitaxel and apoB containing lipoprotein particle may have greater therapeutic efficacy with lower toxicity than current approaches. While this strategy was explored initially for cell-type specific delivery of paclitaxel to HER2-positive breast tumor cells, the approach is a general one that may have broad applicability to other cancers and diseases characterized by surface expression of disease-specific markers.

### EXAMPLE 1

### Expression and Purification of ApoB17F

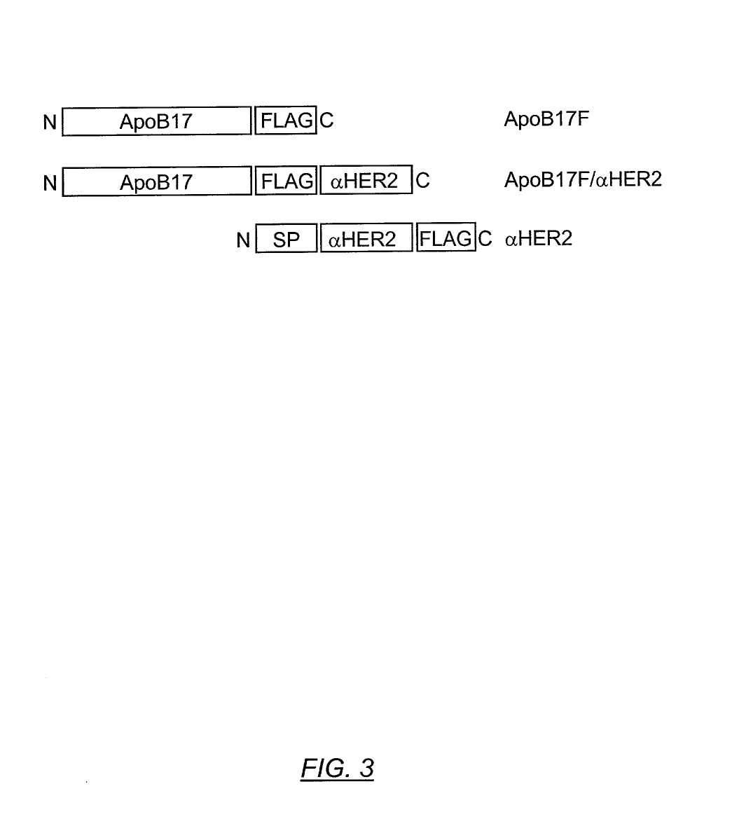

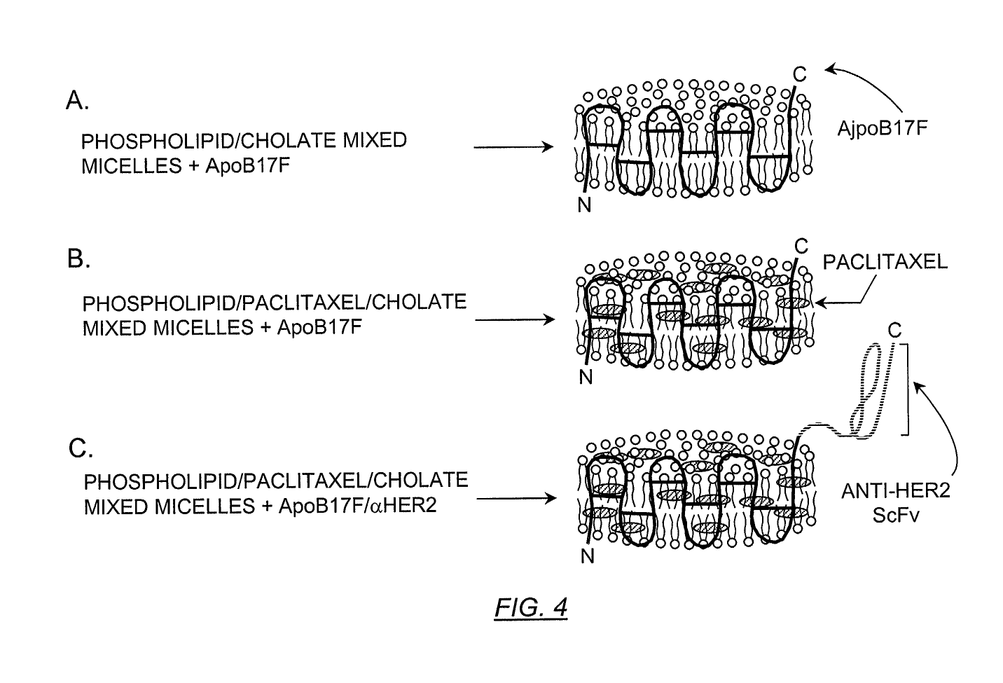

In addition to its lipid sequestration capabilities, apoB17 is readily modifiable by genetic engineering to contain targeting information. For the purpose of the present invention, apoB17 was modified at its C-terminal end to include an anti-HER2 single-chain antibody (apoB17F/αHER2; FIG. 3). The resulting bifunctional protein both compartmentalizes polar lipids and paclitaxel and achieves cell-specific targeting of the protein-lipid-drug complexes to transformed cells (FIG. 4). Previous studies have shown that the simultaneous administration of paclitaxel and anti-HER2 antibody displays greater efficacy than either agent administered alone (Slamon, et al. (1998) Proc. Am. Soc. Clin. Oncol. 17: 98a.(Abstract); Baselga, et al. (1998) Cancer Res. 58: 2825-2831).

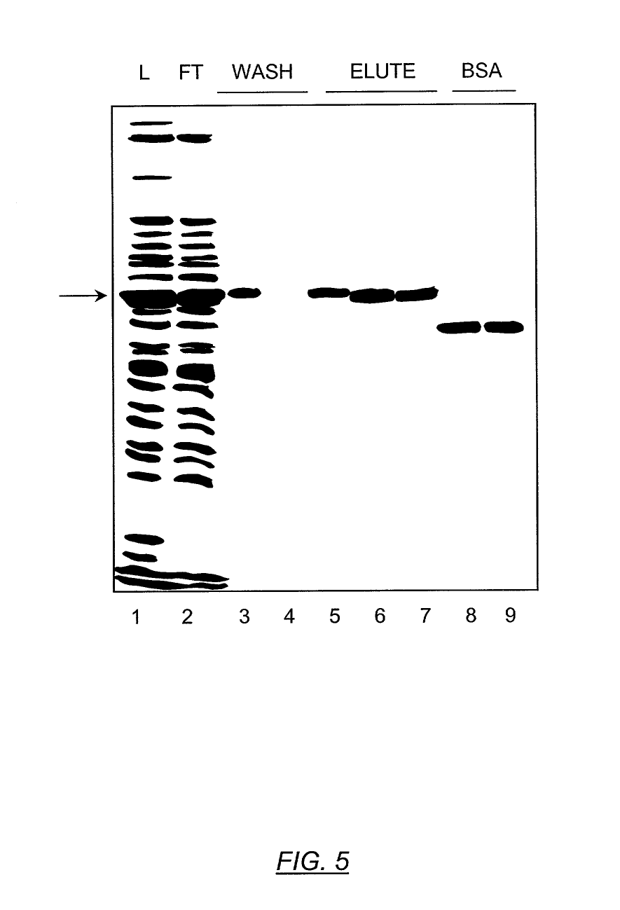

ApoB17F (amino-terminal 17% of apoB followed by 8-amino acid FLAG epitope; FIG. 3) was expressed in Sf9 cells using recombinant baculovirus. Media was harvested and subjected to anti-FLAG affinity chromatography with M2-agarose (Brizzard, et al. (1994) BioTechniques 16:730-734) (FIG. 5). Highly purified protein was eluted from the antibody column using PBS supplemented with 150 μg/mL FLAG peptide (FIG. 5, lanes 5-7). Under the conditions employed, the amount of apoB17F present in the viral supernatant greatly exceeded the binding capacity of the column (FIG. 5, compare load (L) and flowthrough (FT) in lanes 1 and 2). Using 2 mL of column bed volume, 1.8 mg of purified apoB17F was obtained; however, the starting supernatant contains in excess of 10 mg/L of protein. The affinity purification procedure is easily scalable allowing most of the expressed protein to be purified in a single round of chromatography.

### EXAMPLE 2

### Lipid Binding Properties of Recombinant ApoB17F.

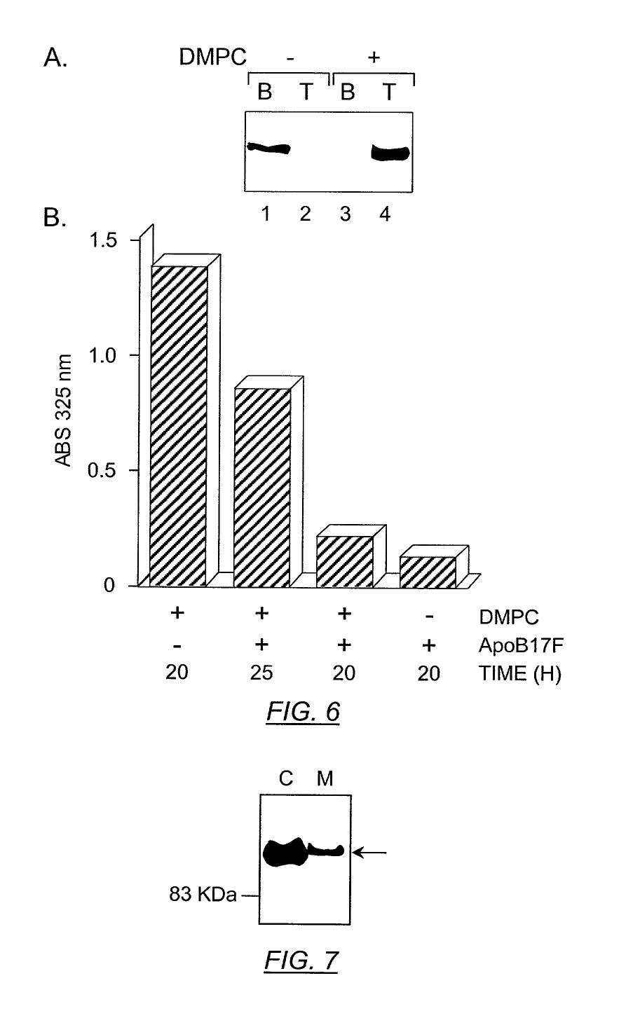

This example demonstrates the strong affinity of apoB17F for polar lipids. The apoB17F (200 μg/mL) purified in FIG. 5 was incubated with and without dimyristoylphosphatidylcholine (DMPC) multilamellar vesicles. Samples were adjusted to 1.25 g/mL with solid KBr and subjected to equilibrium density gradient ultracentrifugation. Without DMPC, all of the apoB was recovered in the lipid-poor bottom (B) fraction (FIG. 6, Panel A, lane 1). Upon incubation with DMPC, the apoB17F associated with the vesicles and floated to the top (T) of the density gradient (FIG. 6, Panel A, lane 4). In FIG. 6, Panel B, the optical density of the vesicle binding reaction was monitored. Upon addition of apoB17F, the optical density of the large multilamellar vesicles was reduced to near background levels (i.e., similar to samples containing apoB17F with no DMPC). This vesicle clearing assay is consistent with the formation of recombinant discoidal lipoproteins (Herscovitz, et al. (1991) Proc. Natl. Acad. Sci. USA 88:7313-7317) (see FIG. 4).

### EXAMPLE 3

### Secretion of ApoB17-Anti-HER2 Single-Chain Antibody from Transfected COS Cells

A fusion protein encoding the amino-terminal 17% of apoB followed by the FLAG epitope and a single-chain antibody directed against HER2 was constructed (FIG. 3, Panel C). The single-chain antibody clone was obtained from Dr. Si-Yi Chen (Baylor College of Medicine). When fused to Pseudomonas exotoxin, this single-chain antibody kills HER2-overexpressing cells (Batra, et al. (1992) Proc. Natl. Acad. Sci. USA 89:5867-5871). The resulting clone was sequenced and found to encode the apoB17F/αHER2 fusion protein as diagrammed in FIG. 3. The cDNA was cloned under the control of the CMV immediate early promoter in plasmid pCMV5 and transfected into COS cells. Twenty-four hours post-transfection, cells were labeled with [³⁵S]Met for 2 h. Cell lysate (C) and media (M) were subjected to immunoprecipitation with anti-FLAG monoclonal antibody and protein G-Sepharose as previously described (Shelness, et al. (1994) J. Biol. Chem. 269:9310-9318). Immune complexes were analyzed by SDS-PAGE and fluorography. The results (FIG. 7) demonstrated a high level of intracellular accumulation and a moderate rate of secretion of a protein with a molecular weight of 110 kDa, consistent with the open reading frame present in apoB17F/αHER2. Proteins that undergo even minimal secretion in short-term metabolic labeling experiments appear to accumulate to high levels during prolonged cell culture. The level of secretion observed here will be adequate to produce large amounts of proteins with the baculovirus Sf9 cell expression system.

### EXAMPLE 4

### Detection and Quantitation of Paclitaxel by Mass Spectrometry

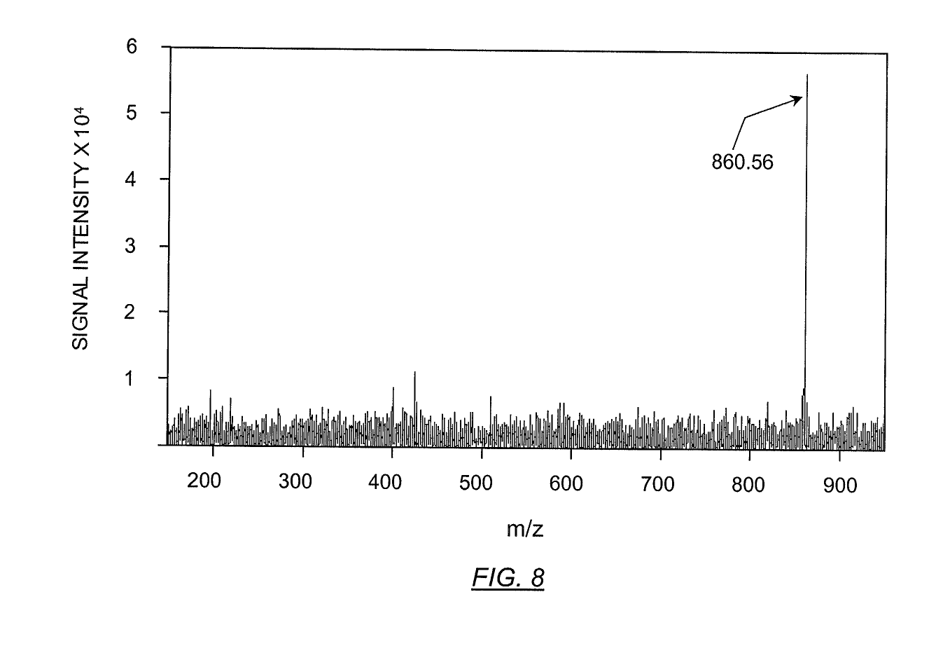

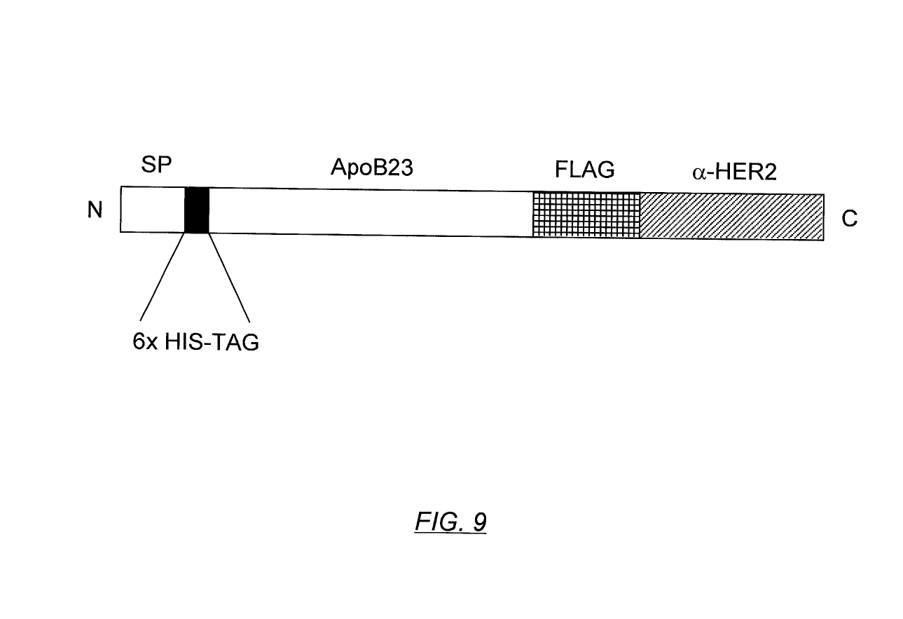

The paclitaxel and lipid content of discoidal lipoproteins was analyzed. To confirm the suitability of this approach, 100 μl of a 100 nM solution of the lithium derivative of paclitaxel was subjected to electrospray mass spectrometry. A peak of m/z 860.56 was readily detected (FIG. 8). Its identity as paclitaxel was confirmed by monitoring a characteristic fragment of m/z 292. As this fragment is unique to paclitaxel and not generated by any other lipid species, there should be no difficulty in detecting the paclitaxel and phospholipid content of recombinant lipoproteins with single electrospray injections. Furthermore, the sensitivity of the assay is extremely high allowing one to easily monitor 10 pMoles (8.6 ng) or less of drug. Conservatively, it is expected that 1 mg of paclitaxel per mg of apoB17F can be compartmentalized.

### EXAMPLE 5

### Production of ApoB17/Anti-HER2 Fusion Protein

The parental apoB17 construct was successfully expressed in baculovirus-infected Sf9 cells and its properties characterized (FIG. 5 and FIG. 6). To examine the feasibility of producing apoB17 fused to a single-chain antibody (apoB17F/αHER2), the fusion protein was constructed and transfected into COS cells. Metabolic labeling followed by immunoprecipitation revealed successful expression and moderate secretion (FIG. 7). To produce biochemical amounts of the fusion protein recombinant baculovirus are used as described above for the apoB17F construct. As a control for single-chain antibody binding, a FLAG epitope-tagged form of the anti-HER2 single-chain antibody is expressed (FIG. 3, lower construct). Using the baculovirus system, milligram quantities of the apoB17 parent protein have been obtained. Furthermore, baculovirus and other insect expression systems have been used successfully to express functional single-chain antibodies and single-chain antibody fusion proteins (Bei, et al. (1995) J. Immun. Meth. 186:245-255; Mahiouz, et al. (1998) J. Immun. Meth. 212:149-160). However, if difficulties recovering sufficient mass from baculovirus supernatant are encountered, one skilled in the art would appreciate several alternative approaches that could be explored. First, the infected Sf9 cells will represent a rich source of recombinant protein. Based on the COS transfection data, the protein appears to be quite stable and accumulates to a significant level (FIG. 7). Methods for the affinity isolation of intracellular forms of apoB17F from stably transfected rat hepatoma cells (McA-RH7777 cells) have been developed. These intracellular forms of apoB17 contain mutations that completely block their secretion. Nevertheless, the affinity purified protein retains its lipid binding function (data not shown). Alternatively, the fusion protein can be expressed in E.coli. While this approach will likely require in vitro folding, there is extensive literature demonstrating the feasibility of producing functional single chain antibodies in this manner (Ge, et al. (1995) In: Antibody Engineering. C. A. K. Borrebaeck, editor.Oxford University Press, New York. 229-266; Huston, et al. (1995) In: Antibody Engineering. C. A. K. Borrebaeck, editor.Oxford University Press, New York. 185-227; Johnson and Bird (1991) Methods Enzymol. 203:88-98; Filpula, et al. (1999) In: Antibody Engineering. A Practical Approach. J. McCafferty, H. R. Hoogenboom and D. J. Chiswell, editors. IRL Press, Oxford. 203-268). While the behavior of the apoB17F moiety within the fusion construct can not be predicted, extensive analyses of disulfide bond formation in this region of apoB have been performed (Shelness and Thornburg (1996) J. Lipid Res. 37:408-419; Ingram and Shelness (1997) J. Biol. Chem. 272:10279-10286; Huang and Shelness (1997) J. Biol. Chem. 272:31872-31876). These studies demonstrated that fully reduced apoB28 can achieve its native disulfide bonds post-translationally (Ingram and Shelness (1997) J. Biol. Chem. 272:10279-10286). Therefore, if necessary, conventional methods of in vitro protein folding can be applied to the production of the bifunctional apoB17-single-chain antibody fusion construct expressed in E. coli.

### EXAMPLE 6

### Formation of Phopholidpid-Taxol-ApoB17 Complexes

The capacity to sequester paclitaxel in recombinant lipoproteins is explored first using the unmodified form of apoB17F. Phospholipid-paclitaxel-apoB17F complexes are formed by cholate dialyses. This procedure allows detergent solubilization of lipophilic substances at a defined molar ratio followed, by dialysis to remove detergent (Jonas (1986) Methods Enzymol. 128:553-582). As a result, recombinant lipoproteins are formed whose composition generally reflects the ratio of components present in the initial cholate micelles. Unreacted or precipitated lipid is removed by centrifugation at 15,000 rpm for 1 h. The concentration of recombinant lipoprotein complexes can be increased by centrifugal dialysis. To examine the relative percentage of apoB17F that complexed with lipid and drug, density gradient ultracentrifugation is performed as described in EXAMPLE 2. ApoB17F recovered from the top gradient fraction, representing the lipid-bound form, is extracted with chloroform-methanol by the method of Bligh and Dyer ((1959) Can. J. Biochem. Physiol. 37:911-917). Paclitaxel is fully extractable under these conditions (data not shown), however control experiments utilizing [¹⁴C]paclitaxel (Sigma Chemical Co.) are used to confirm efficient extraction under these conditions. Extracts are quantitated for paclitaxel and lipid mass by electrospray mass spectrometry. ApoB is quantified by BCA protein assay (Pierce). The relative ratio of components packaged into lipoprotein particles is calculated.

ApoB17 forms discoidal particles with an estimated composition of 2250 molecules of phosphatidylcholine per 2 molecules of apoB17 (Herscovitz, et al. (1991) Proc. Natl. Acad. Sci. USA 88:7313-7317). Free cholesterol, a compound with solubility properties similar to paclitaxel, is soluble in phospholipid disks at a value of ˜30 mole % (Jonas (1986) Methods Enzymol. 128:553-582). Assuming hypothetically that these particles accommodate 23 mole % paclitaxel, 1 mg of apoB will sequester 3.4 mg of paclitaxel. However these values will vary depending upon the starting ratio of components in the dialysis mixture as well as the species of lipid employed. The ratio of components as well as the effects of different fatty acyl species are systematically explored to develop optimal conditions for solubilization of paclitaxel. Even if only 5-10% solubility is achieved, these complexes will still be therapeutically useful. Once optimal conditions are established for generation of paclitaxel-enriched apoB17F lipoprotein particles, the apoB17/αHER2 construct is tested. While it is conceivable that the single-chain antibody will disrupt the lipid binding property of apoB17 or vice versa, previous studies have demonstrated that the amino terminal 17% of apoB folds independently of the rest of the apoB protein (Ingram and Shelness (1997) J. Biol. Chem. 272:10279-10286). It is anticipated that this domain will continue to function as a strong lipid binding protein even when expressed as a fusion with a single-chain antibody.

### EXAMPLE 7

### Interaction of ApoB17F/αHER with HER2 Overexpressing Cells

To test the single-chain antibody's capacity to target the fusion constructs, their capacity to react with cell surface HER2 is assessed. Recombinant protein is incubated with MCF7 and MDA-231 cells (low HER2-expressing controls; 7.27 and 5.40 ng/mg cell protein, respectively) and SKBR3 and BT474 cells (HER2-overexpressing breast tumor cells; 917 and 548 ng/mg cell protein, respectively) (Lewis, et al. (1993) Cancer Immunol. Immunother. 37:255-263). Cell monolayers are incubated with recombinant protein (1-50 μg/mL) at 4° C. After washing with PBS, cells are fixed with formalin and incubated with FITC-conjugated anti-FLAG monoclonal antibody M2. FITC-labeled M2 is available commercially from Sigma Chemical Co. Previous studies have demonstrated the efficacy of monoclonal antibody M2 for both immunoperoxidase staining (Shelness (1992) IBI FLAG Epitope. A Technical Bulletin for Users of FLAG Technology 1:2-3) and immunofluorescence (data not shown). Both naked protein and protein assembled with lipid are compared. A control single-chain antibody against HER2 (FIG. 3) (Batra, et al. (1992) Proc. Natl. Acad. Sci. USA 89:5867-5871) as well as Herceptin™ are used to establish the upper limit of staining intensity. Herceptin™ is supplied by Dr. Tim Kute who has obtained the antibody from Genentech for research purposes. These studies are well controlled and readily interpretable. Not only is fluorescence intensity compared in both low and high HER2-expressing cells but a comparison is made between HER2-overexpressing cells incubated with the apoB17F parent construct, which contains no targeting information (i.e., negative control), and the apoB17-single-chain antibody fusion (apoB17F/αHer2). Subsequently, both the apoB17F and apoB17F/αHER2 proteins are lipidated with phosphatidylcholine and the experiments repeated to confirm that lipidation has no impact on single-chain antibody function.

To specifically access the extent of internalization, cells are incubated with the various proteins and protein-lipid complexes. As a means of promoting a synchronized burst of endocytosis, cells are incubated at 4° C. for 1-3 h to promote cell surface binding and then shifted to a humidified, 37° C. incubator containing 5% CO₂ for various times from 1-24 h (Maier, et al. (1991) Cancer Res. 51: 5361-5369; Landgraf, et al. (1998). Biochemistry 37: 3220-3228). Cells are treated with 0.2 N acetic acid for 6-12 min at room temperature to remove cell-associated, non-internalized proteins. Cells are formalin-fixed and permeabilized with 0.1% Triton X-100 followed by staining with anti-FLAG monoclonal antibody M2. A quantitative assessment of the rate and extent of internalization is performed by incubation of cells with iodinated protein or protein-lipid complex (Landgraf, et al. (1998). Biochemistry 37: 3220-3228; Bilheimer, et al. (1972) Biochim. Biophys. Acta 260: 212-221). After incubation at 4° C., cells are transferred to a 37° C. incubator for various times up to 24 h. After each period, cells are placed on ice and the media removed. Cells are washed with PBS and incubated with PBS containing 100 μg/mL proteinase K for 1 h at 4° C. The PBS is removed and centrifuged to harvest dislodged cells. The counts in supernatant represent bound, noninternalized protein. The dislodged cells and the cells remaining on the plate (internalized fraction) are solubilized and counted. Counts obtained in the presence of a 500-fold excess of cold test protein or complex are subtracted. It is assumed that the counts present in the initial supernatant represent unbound protein. However, if extensive cell-mediated degradation occurs, soluble counts are released into the media. To assess the degree to which counts present in media represent cell-mediated degradation of internalized protein, trichlroaceteic acid (TCA) precipitation is performed. TCA soluble counts in media are considered the product of cell-associated protein degradation; however controls are performed with conditioned media to ensure that degradation is not due to secretion of proteases.

### EXAMPLE 8

### Anti-Proliferative Effects of ApoB-Anti HER2 Fusion Proteins in Monolayer Cultures

The anti-proliferative effects of the recombinant proteins both in naked form and complexed with lipids and paclitaxel are assessed by clonagenic assay and/or cell counting. Control and HER2 overexpressing cell lines are plated in 60 mm dishes at low density (200-800 cells per dish). After addition of experimental media, cells are incubated for 5-7 days, washed, fixed in 10% formalin, rinsed in PBS, and stained with 0.1% crystal violet. Crystal violet-positive colonies are counted manually. Alternatively, after incubation with experimental media, cells are trypsinized and counted manually or using a Coulter cell counter.

In the initial round of experiments, the effects of the naked protein constructs is assessed. Both control and HER2-overexpressing cells are incubated with media containing 0.5% fetal bovine serum and one of the following additions: (1) 5, 10, and 25 nM irrelevant monoclonal antibody; (2) 5, 10, and 25 nM apoB17F; (3) 5, 10, and 25 nM apoB17F/αHER2; (4) 5, 10, and 25 nM Herceptin™ (Baselga, et al. (1998) Cancer Res. 58: 2825-2831). Experimental media is replaced every 2-3 days. These studies establish whether the apoB-single-chain fusion protein display antiproliferative effects independently of its association with lipid and drug and the potency of these effects relative to Herceptin™. Comparison with the lipidated forms of the recombinant proteins determine whether association with phospholipid in any way modulates the inherent anti-proliferative effects of the apoB or apoB-single-chain antibody fusion.

### EXAMPLE 9

### Anti-Proliferative Effects of Paclitaxel Delivered to Control and HER2 Overexpressing Cells in Targeted and Nontargeted Recombinant Lipoproteins

In subsequent studies, the effects of delivering paclitaxel in the form of recombinant lipoproteins is assessed. In these experiments cells are incubated with media containing (1) apoB17F-phopholipid complex; (2) apoB17F-phospholipid-paclitaxel complex; or (3) apoB17/αHER2-phospholipid-paclitaxel complex. The amount of paclitaxel compartmentalized into the apoB17F complexes is determined empirically using mass spectrometry (FIG. 8). The mass of fusion protein added to the cells depends on the amount of paclitaxel compartmentalized per complex. Ideally, the paclitaxel:protein ratio can be controlled sufficiently to simultaneously deliver the proper range of both drug and apoB17F/αHER2 fusion protein necessary to inhibit monolayer cell growth (Baselga, et al. (1998) Cancer Res. 58: 2825-2831). As a positive control, cells are treated with media containing an anti-proliferative dose range of paclitaxel (0-25 μM) with and without 5 nM Herceptin™ (Baselga, et al. (1998) Cancer Res. 58: 2825-2831). Generally, a 1 h incubation with paclitaxel is sufficient to exert anti-proliferative effects, whereas the effect of Herceptin™ on cultured cells requires incubation throughout the experimental period (Baselga, et al. (1998) Cancer Res. 58: 2825-2831). In the case of the recombinant lipoproteins, the paclitaxel and single-chain antibody remains throughout the incubation as the two functions cannot be separated. However, the apoB-lipid-drug complexes may be removed after a shorter term incubation period if necessary.

The no drug control (apoB17F-phospholipid complex) and the paclitaxel+Herceptin™ treated cells (positive control) provide the two extremes of cell or colony number. If paclitaxel remains stably compartmentalized in the apoB17F particles, then little reduction in cell or colony number will be observed upon incubation with the apoB17F-lipid-paclitaxel complex. Addition of the apoB17F/αHER2-containing particle may result in drug targeting in HER2 expressing cells but not in control cells expressing low levels of HER2. Hence, it is expected that a greater attenuation of cell growth in the former group will be observed. These experiments establish whether (1) paclitaxel compartmentalized within recombinant lipoprotein is relatively inert unless modified with the single-chain antibody targeting domain and (2) that the anti-proliferative effects demonstrated by these complexes is enhanced in HER2-coverexpressing cells relative to control cells.

### EXAMPLE 10

### Assay of Tumor Growth in Athymic Nude Mice

Initial characterization of the experimental complexes are performed using in vitro assays to demonstrate the ability of the experimental complexes to inhibit the proliferation of transformed cells in culture. However, to demonstrate that these experimental treatments have potential as anti-breast cancer therapy in humans, an animal model of human breast cancer needs to be tested. Growth of tumor xenografts in immuno-compromised mice represents the most common system in which these preliminary studies are performed. It is for this reason that the athymic nude mouse system is used. The numbers of animals to be used are modeled after studies in the literature detailing the synergistic effects of anti HER2 antibody (Herceptin™) and paclitaxel. As the experimental drugs described herein combine these two treatments into a single targeted entity, it is assumed that similar animal group sizes would be required to achieve adequate statistical power.

Five-Seven week old, Balb/c, female athymic nude mice are used as hosts for growth of HER2 expressing tumor xenografts. These animals are used to assess the therapeutic effects of the paclitaxel-anti-HER2 complexes. The animals are injected subcutaneous with 100 μL of HER2-overexpressing cells (1-2×10⁶ cells) to initiate tumor formation. As the tumors grow subcutaneously, they are easily measured using calipers. The growth of tumors are monitored over a 4-6 week period as a function of both control treatments and treatments with the experimental drug-antibody complexes. The animal receives the experimental treatments by inter-peritoneal injections. This mode of administration may provide sufficient absorption of these complexes. However, if necessary, compounds may be administered by tail vein injection or slow retro-orbital injection. For the control injection of paclitaxel as a cremophor-ethanol emulsion, IV injections must be employed. Again, these are by tail vein or slow retro-orbital injection. During the first phase of these studies 7 experimental groups of 9 animals each (63 total animals) are used. To repeat or modify the experimental design, an additional 4-7 groups of 9 animals are required. Hence, 99-126 animals may be used during the course of these experiments.

The effects of the drug-containing recombinant lipoproteins are extended to an animal model of breast cancer. As described above lower mammary pads of female BALB/c nude mice are injected with SKBR-3 cells expressing high levels of HER2 and are growth inhibited by anti-HER2 antibodies (Lewis, et al. (1993) Cancer Immunol. Immunother. 37:255-263). Every 2-3 days tumor size is measured with vernier calipers and tumor size calculated by the formula (π/6)(larger diameter)(smaller diameter)². When average tumor volumes reach 0.2-0.3 cm³, animals are randomized into treatment groups of 9 animals each (Baselga, et al. (1998) Cancer Res. 58: 2825-2831).

Tumor volumes of treatment groups are expressed as means±standard error (SE). Changes in mean tumor volumes among different groups over the treatment period are analyzed by repeated measures ANOVA (analysis of variance). Comparison of groups at the completion of each treatment regimen are analyzed by ANOVA with Fisher's post-hoc test to tell which groups are different. Statview software are employed for statistical analyses.

Initially, four treatment groups are employed: (1) No drug control group receives ApoB17 recombinant phospholipid disks lacking paclitaxel; (2) Untargeted paclitaxel group receives paclitaxel-enriched apoB17F phospholipid disks; (3) Targeted paclitaxel group receives the paclitaxel enriched phospholipid disks formed with the apoB17/αHER2 fusion protein; (4) paclitaxel positive control group receives injections of paclitaxel. The initial mass of recombinant disks injected are dictated by their paclitaxel content as described in EXAMPLE 6. Assuming, conservatively that disks can be formed containing 10 mole % paclitaxel, then 1 mg of apoB will carry ˜1 mg of paclitaxel. As two, 10 mg/kg injections of paclitaxel on days 1 and 4 exhibits maximal inhibition of tumor growth in nude mice breast tumor xenografts (Baselga, et al. (1998) Cancer Res. 58: 2825-2831), 200 μg of apoB-recombinant phospholipid disk would be required for each injection assuming a mouse weight of 20 g. The phospholipid and phospholipid-paclitaxel complexes are administered by IP injection. Although, paclitaxel is usually administered in mice by retro-orbital injection, this is because of its compartmentalization as a cremophor emulsion (Baselga, et al. (1998) Cancer Res. 58: 2825-2831). These emulsion particles are presumably quite large limiting their entry into capillaries or the lymphatic system after IP injection. The discoidal structure, however, are considerably smaller and may be readily absorbed. Blood samples are obtained in a control mouse using retro-orbital or tail vein bleeds to establish plasma concentrations of injected recombinant disks. To achieve adequate plasma levels of the complexes, the relatively small volumes of lipoproteins are administered by tail vein injection in normal saline, if necessary, or by slow retro-orbital injection in cremophor-ethanol as described (Baselga, et al. (1998) Cancer Res. 58: 2825-2831).

As this therapeutic approach is novel, it is difficult to predict its many potential outcomes. It is presumed that administration of the drug-free recombinant disk (group 1) will produce no inhibition of tumor growth giving rise to mean tumor volumes in the range ˜15-25 cm³ over the 4-6 week experimental period (Baselga, et al. (1998) Cancer Res. 58: 2825-2831). Should the paclitaxel remain compartmentalized within the untargeted apoB17F recombinant disk (group 2), little therapeutic benefit will be observed. However, if the drug exchanges to any extent onto albumin (Beck et al. (1997) In: Cancer Medicine, Volume One. J. F. Holland, R. C. Bast, Jr., D. L. Morton, E. Frei, III, D. W. Kufe and R. R. Weichselbaum, editors. Williams & Wilkins, Baltimore. 1005-1025) or other acceptors in the plasma compartment, tumor growth inhibition will likely be observed. The question of whether the disk is removed from the circulation en block or whether it is remodeled prior to clearance is addressed in the final set of experiments outlined below. In any case, it is anticipated that the apoB17F/αHER2 targeted drug complex will display the greatest inhibition of tumor growth. Such an effect could be due to (1) the targeting of the complex to HER2-expressing cells and cellular uptake of the complex, (2) Herceptin™-like effects of the single-chain antibody, or (3) synergism between the single-chain antibody and the paclitaxel. Should the targeted paclitaxel complex display efficacy over and above the untargeted control, how much of the effects are due to the single-chain antibody versus the delivery of the drug will be examined. For these experiments a second group of tumor-bearing animals are subjected to the following treatments: (1) apoB17 phospholipid complex (no paclitaxel), (2) apoB17/αHER2 phospholipid complex (no paclitaxel), or (3) apoB17/αHER2 phospholipid+paclitaxel complex. This study defines the relative contribution of the single-chain antibody moiety of recombinant disks, independently of drug (i.e. group 2), versus the therapeutic effect of the single-chain antibody in combination with paclitaxel (group 3).

After establishing the relative therapeutic efficacies of the control and drug-containing complexes, additional experiments are designed to examine issues such as dose requirements, including drug:lipid ratios, and dosing schedules. In particular, dosing schedules may be modified as the two-dose regimen is based on administration of paclitaxel alone, whereas Herceptin™ is generally administered twice per week for the entire experimental period of ˜6 weeks (Baselga, et al. (1998) Cancer Res. 58: 2825-10 2831). Should less than optimal inhibition of growth be observed using the day 1 and 4 regimen, the treatments are lengthened to included twice-weekly injections throughout the monitoring period. Conversely, because of the targeting sequences present in the apoB17F/αHER2 construct, a much lower concentrations of paclitaxel may be required to achieve the same therapeutic effect observed for injection of paclitaxel as the cremophor-ethanol emulsion.

### EXAMPLE 11

### Turnover and Remodeling of Lipid-drug Complexes

A question with significant implications for the potential efficacy of the apoB-lipid/drug complexes as therapeutic agents relates to their half-lives in plasma and the extent to which they undergo intravascular remodeling. In the human genetic disorder, familial hypobetalipoproteinemia, frameshift mutations generate variously sized truncated forms of apoB (Linton, et al. (1993) J. Lipid Res. 34:521-541). However, mutations resulting in truncations smaller than the amino terminal ˜25% create unstable mRNAs. This severe biosynthetic defect results in the absence of these small, truncated products in plasma (Srivastava, et al. (1999) J. Lipid Res. 40:901-912). Therefore, there is little current information on the metabolism of very small truncated forms of apoB either as naked proteins or when packaged into recombinant lipoproteins. It was recently demonstrated that injection of mice with recombinant adenoviruses engineered to express high levels of apoB17, results in apoB17 plasma concentrations 24-fold higher than endogenous mouse apoB (Li et al. (2000) J. Lipid Res. 41:1912-1920). Hence, it would appear that small amino-terminal forms of apoB are not inherently labile.

To more systematically explore this issue, 1 mg of apoB17 recombinant lipoprotein is injected into the tail veins of female Wistar rats (N=3). In this case rats are chosen because of their larger sizes and blood volumes which facilitate the multiple blood samplings that are required for turnover studies. Groups of three animals are employed for these studies in order to generate a mean and standard deviation for each time point during the turnover study. These studies are performed using both the control complex containing lipid with no drug as well as the paclitaxel-lipid complexes. Hence, 3 studies with 3 animals each are performed. Blood samples (1 mL) are drawn from the tail vein at 0, 1, 4, 8, and 24 h post-injection. Plasma samples are analyzed by a sensitive sandwich ELISA assay that has been developed for FLAG-tagged forms of apoB, including apoB17. The apoB17 mass concentration is assessed using apoB17F standards. Subsequent time courses of turnover are modified depending on the outcome of this initial analysis. The studies are also repeated using apoB17F-lipid-paclitaxel complexes. In these studies, both protein turnover and the paclitaxel and lipid content of the particles are analyzed. For this purpose particles are immuno-affinity purified with anti-FLAG-agarose and the lipids and paclitaxel are extracted with methanol-chloroform, as described (Bligh and Dyer (1959) Can. J. Biochem. Physiol. 37: 911-917). The lipid:paclitaxel ratio is determined by electrospray mass spectrometry and normalized to protein content based on the ELISA assay. These studies determine whether the paclitaxel content of the complexes is relatively stable or whether the paclitaxel exchanges readily to other acceptors such as albumin or endogenous lipoproteins.

### EXAMPLE 12

### Design, Construction, Expression, and Purification of an ApoB23-anti-HER2 Single-Chain Antibody Fusion Protein

A recombinant protein consisting of apoB23 fused to an anti-HER2 single-chain antibody was designed (FIG. 9). The protein fusion was constructed using overlap PCR extension techniques known to one skilled in the art. The fusion protein was expressed in the expression plasmid pCMV5 under the control of the CMV immediate early promoter. This protein contains a 6× His-tag after the apoB signal peptide, followed by apoB23, FLAG epitope tag, and anti-HER2 single-chain antibody (FIG. 9).

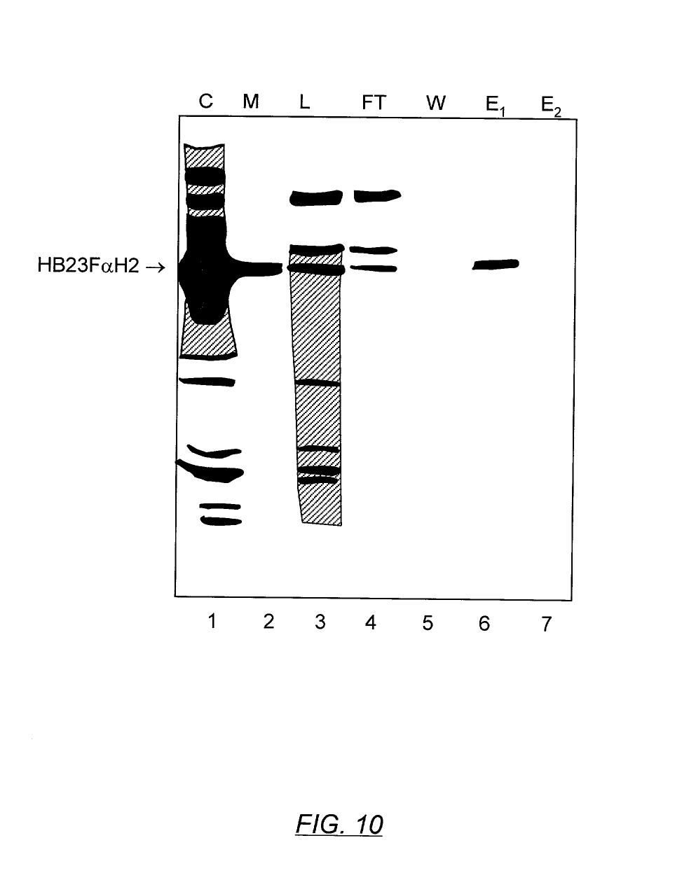

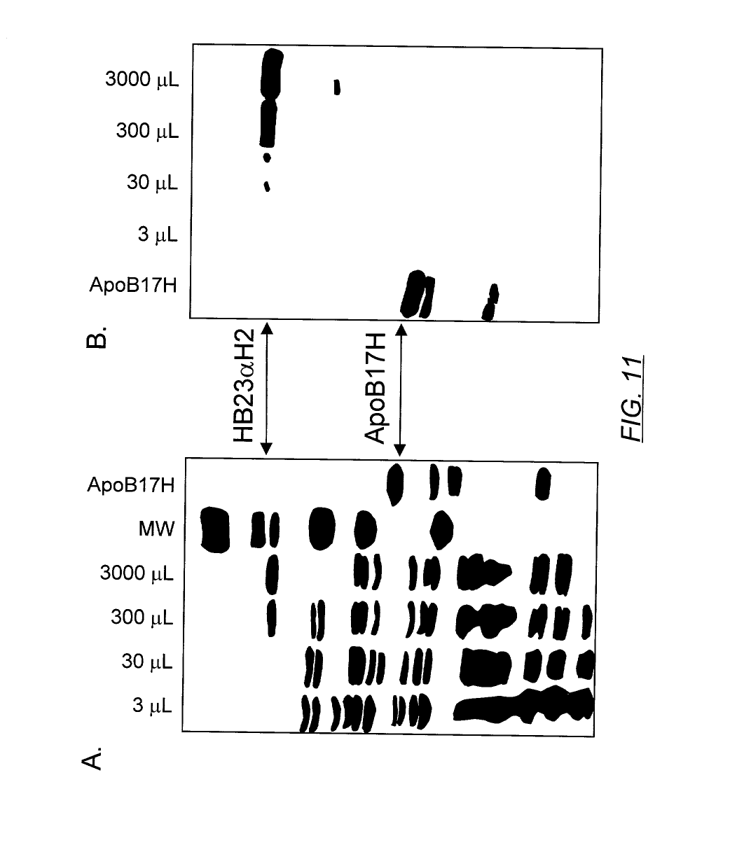

COS-1 cells were transfected with pCMV5 plasmid harboring HB23αH2. After transfection, cells were radiolabeled with [³⁵S]Met/Cys for 3 h. Subsequently, media and cellular material were subjected to immunoprecipitation with anti-apoB antibodies (FIG. 10, lanes 1 and 2). The medium was also subjected to nickel chromatography (FIG. 10, 3-7) and subsequent imidazole elutions (FIG. 10, lanes 6 and 7). Protein samples were separated by SDS-polyacrylamide gel electrophoresis and visualized by fluorography. Note that the fusion protein was secreted with relatively high efficiency from transfected COS-1 cells and was eluted in pure form from the nickel column (FIG. 10, Lane 6). The protein was also expressed using the baculovirus system and was shown to accumulate in recombinant baculovirus Sf9 cell supernatant to a level of approximately 1 mg/L (FIG. 11).

The foregoing examples are illustrative of the present invention, and are not to be construed as limiting thereof. The invention is described by the following claims, with equivalents of the claims to be included therein.

What is claimed is:

1. A lipoprotein compound delivery particle, comprising:
(a) from 0.1 to 90 percent by weight of a lipophilic or amphipathic compound to be delivered;
(b) from 0 to 50 percent by weight of at least one polar lipid in an amount sufficient to form a particle with said lipophilic compound
(c) from 0 to 90 percent by weight of at least one neutral lipid; and
(d) from 0.5 to 90 percent by weight of a truncated apolipoprotein B protein in said particle having a deleted LDL receptor binding region.

2. The particle according to claim 1, wherein said apolipoprotein B further comprises a fused heterologous moiety, where said heterologous moiety is a member of a specific binding pair.

3. The particle according to claim 2, wherein said heterologous moiety is a peptide.

4. The particle according to claim 2, wherein said heterologous moiety is an antibody.

5. The particle according to claim 2, wherein said heterologous moiety is a single chain antibody.

6. The particle according to claim 2, wherein said heterologous moiety is a single chain anti HER2 antibody.

7. The particle according to claim 1, wherein said particle has a diameter less than 18 nanometers.

8. The particle according to claim 1, wherein said particle has a diameter of from 5 to 5,000 nanometers.

9. The particle according to claim 1, wherein said said apolipoprotein B is selected from the group consisting of through apoB74.

10. The particle according to claim 1, wherein said particle has a neutral core, and wherein said ApoB comprises at least ApoB 19.5.

11. The particle according to claim 1, wherein said apolipoprotein B is mature Apo B.

12. The particle according to claim 1, wherein said apolipoprotein B is mammalian Apo B.

13. The particle according to claim 1, wherein said apolipoprotein B is human Apo B.

14. The particle according to claim 1, wherein said at least one polar lipid is a phosphatidylcholine, phosphatidylethanolamine, phosphatidylserine, phosphatidylinositol, sphingomyelin, glycosphingolipid, lysolipid thereof, or combinations thereof.

15. The particle according to claim 1, wherein wherein said at least one neutral lipid comprises a triglyceride, cholesterol, derivative thereof, or combination thereof.

16. The particle according to claim 1, wherein said compound to be delivered is paclitaxel.

17. The particle according to claim 1, comprising:
(a) from 0.1 to 50 percent by weight of said compound to be delivered;
(b) from 10 to 50 percent by weight of said at least one polar lipid;
(c) from 0 to 10 percent by weight of at said least one neutral lipid; and
(d) from 50 to 90 percent by weight of said truncated apoB.

18. The particle according to claim 17, wherein said particle is a discoidal particle.

19. The particle according to claim 1, comprising:
(a) from 0.1 to 55 percent by weight of said compound to be delivered;
(b) from 15 to 55 percent by weight of said at least one polar lipid;
(c) from 2 to 30 percent by weight of at said least one neutral lipid; and
(d) from 30 to 80 percent by weight of said truncated apoB.

20. The particle according to claim 19, wherein said particle is a small emulsion particle.

21. The particle according to claim 19, comprising:
(a) from 0.1 to 80 percent by weight of said compound to be delivered;
(b) from 1 to 30 percent by weight of said at least one polar lipid;
(c) from 30 to 90 percent by weight of at said least one neutral lipid; and
(d) from 0.5 to 10 percent by weight of said truncated apoB.

22. The particle according to claim 21, wherein said particle is a large emulsion particle.

23. The particle according to claim 21, wherein said compound to be delivered is an amphipathic compound, and wherein said amphipathic compound comprises a synthetic lipid.

24. A pharmaceutical formulation comprising a plurality of lipoprotein compound delivery particles of claim 1.

25. The pharmaceutical formulation of claim 24, consisting essentially of said particles in a size of 2 to 20 nanometers in diameter.

26. The pharmaceutical formulation of claim 24, consisting essentially of particles in a size of 5 to 40 nanometers in diameter.

27. The pharmaceutical formulation of claim 24, consisting essentially of particles in a size of 10 to 60 nanometers in diameter.

28. The pharmaceutical formulation of claim 24, consisting essentially of particles in a size of 15 to 100 nanometers in diameter.

29. The pharmaceutical formulation of claim 24, consisting essentially of particles in a size of 25 to 200 nanometers in diameter.

30. The pharmaceutical formulation of claim 24, consisting essentially of particles in a size of 50 to 1,000 nanometers in diameter.

31. The pharmaceutical formulation of claim 24, consisting essentially of particles in a size of 250 to 5,000 nanometers in diameter.

32. The pharmaceutical formulation of claim 24, in a pharmaceutically acceptable carrier.

33. The pharmaceutical formulation of claim 32, wherein said carrier is an aqueous carrier.

34. The pharmaceutical formulation of claim 24, in sterile lyophilized form.

35. A method of delivering a compound to a subject in need thereof, comprising administering a lipoprotein compound delivery particle of claim 1 to said subject in an amount effective to deliver said compound to said subject.

36. The method according to claim 35, wherein said administering step is carried out by parenteral injection.

37. The method according to claim 35, wherein said administering step is carried out by intraveneous injection.

38. The method according to claim 35, wherein said administering step is a topical administration step.

39. A covalent conjugate, comprising:
(a) a truncated apolipoprotein B protein in having a deleted LDL receptor binding region; covalently coupled to
(b) a heterologous moiety, where said heterologous moiety is a member of a specific binding pair.

40. The compound according to claim 39, wherein said conjugate is a fusion protein.

41. The compound according to claim 39, wherein said truncated apolipoprotein B is selected from the group consisting of apoB6 through apoB74.

42. The compound according to claim 39, wherein said heterologous moiety is a receptor binding group.

43. The compound according to claim 39, wherein said compound is an apoB23 anti-HER2 single-chain antibody fusion protein.
